(19) United States
(12) Patent Application Publication
(10) Pub. No.: US 2025/0347616 A1
(43) Pub. Date: Nov. 13, 2025

(54) DETECTION DEVICE, OPTICAL SYSTEM INCLUDING THE SAME, AND METHOD FOR DETECTING CONCENTRATION USING THE SAME

(71) Applicant: VisEra Technologies Company Limited, Hsin-Chu City (TW)

(72) Inventors: Hsin-Yi HSIEH, Hsin-Chu City (TW); Chia-Chun CHANG, Hsin-Chu City (TW)

(21) Appl. No.: 18/941,011

(22) Filed: Nov. 8, 2024

Related U.S. Application Data
(60) Provisional application No. 63/646,183, filed on May 13, 2024.

(51) Int. Cl. G01N 21/01; G01N 21/21; G01N 21/47; G02B 5/30

(52) U.S. Cl. CPC G01N 21/01; G01N 21/21; G01N 21/47; G02B 5/3025; G01N 2021/0106; G01N 2021/216; G01N 2021/4709; G01N 2333/46

(57) ABSTRACT

A detection device is provided. The detection device includes a substrate having a first photoelectric conversion element and a second photoelectric conversion element. The detection device also includes a first filter disposed above the first photoelectric conversion element and a second filter disposed above the second photoelectric conversion element. The first photoelectric conversion element collects light with the same polarization direction as the first circularly polarized light, and the second photoelectric conversion element collects light with opposite polarization direction to the first circularly polarized light.

### CROSS REFERENCE TO RELATED APPLICATIONS

This application claims the benefit of U.S. Provisional Application No. 63/646,183, filed on May 13, 2024, the entirety of which is incorporated by reference herein.

### BACKGROUND OF THE INVENTION

### Field of the Invention

The present disclosure relates to a detection device, and, in particular, to a detection device for detecting a backscattering light, an optical system including the same, and a method for detecting the concentration of a specific substance in a biosample using the same.

### Description of the Related Art

Integrated sensing devices have recently become popular for use in biological analysis. For example, a traditional detection device may be implanted within a living organism (e.g., a human) and used to measure an analyte (e.g., glucose) in a medium (e.g., interstitial fluid (ISF), blood, or intraperitoneal fluid) within the living organism. However, traditional detection devices often require intrusion into living organisms, which may cause discomfort. Alternately, the sensing results by the traditional detection device may have large errors due to weak sensing signals.

Although existing detection devices have been adequate for their intended purposes, they have not been entirely satisfactory in all respects. Therefore, a novel detection device is still in demand.

### BRIEF SUMMARY OF THE INVENTION

In the embodiments of the present disclosure, the detection device includes at least two photoelectric conversion elements to collect different circularly polarized light, thereby enhancing the detection capabilities in diverse optical environments. The detecting method in the embodiment of the present disclosure on-invasive, which may be designed for applications in glucose sensing and tumor imaging via tissue slices or endoscopes.

An embodiment of the present invention provides a detection device for detecting a backscattering light BL that is generated after a first circularly polarized light is emitted into a biosample. The detection device includes a substrate having a first photoelectric conversion element and a second photoelectric conversion element. The detection device also includes a first filter disposed above the first photoelectric conversion element and a second filter disposed above the second photoelectric conversion element. The first photoelectric conversion element collects light with the same polarization direction as the first circularly polarized light, and the second photoelectric conversion element collects light with opposite polarization direction to the first circularly polarized light.

In some embodiments, the detection device further includes a cover plate disposed over the first filter and the second filter.

In some embodiments, the detection device further includes a light-condensing structure disposed on the cover plate.

In some embodiments, the light-condensing structure includes microlenses or metalens.

In some embodiments, the detection device includes a first linear polarizer disposed above the first photoelectric conversion element. The detection device also includes a second linear polarizer disposed above the second photoelectric conversion element. The second linear polarizer is shifted 90 degrees from the first linear polarizer. The detection device further includes a quarter-wave plate disposed above the first linear polarizer and the second linear polarizer. Moreover, the detection device includes a light-transmitting layer disposed between the first linear polarizer or the second linear polarizer and the quarter-wave plate. The first linear polarizer, the light-transmitting layer, and the quarter-wave plate form the first filter, and the second linear polarizer, the light-transmitting layer, and the quarter-wave plate form the second filter.

In some embodiments, the detection device further includes a first light source disposed on the substrate and a first polarization light-converting structure disposed above the first light source. The first circularly polarized light is generated from the first light source through the first polarization light-converting structure.

In some embodiments, the first polarization light-converting structure includes metasurface.

In some embodiments, the first polarization light-converting structure includes a linear polarizer and a quarter-wave plate disposed above the linear polarizer. Moreover, the first polarization light-converting structure further includes a light-transmitting layer disposed between the linear polarizer and the quarter-wave plate.

In some embodiments, the detection device further includes a second light source disposed on the substrate and a second polarization light-converting structure disposed above the second light source. A second circularly polarized light having a different wavelength than the first circularly polarized light is generated from the second light source through the second polarization light-converting structure.

In some embodiments, the second polarization light-converting structure includes metasurface.

In some embodiments, the second polarization light-converting structure includes a linear polarizer and a quarter-wave plate disposed above the linear polarizer. Moreover, the second polarization light-converting structure further includes a light-transmitting layer disposed between the linear polarizer and the quarter-wave plate.

In some embodiments, there are a plurality of first photoelectric conversion elements and a plurality of second photoelectric conversion elements that are arranged in an array, and there are a plurality of first filters disposed above the first photoelectric conversion elements and a plurality of second filters disposed above the second photoelectric conversion elements.

In some embodiments, the substrate further has a third photoelectric conversion element adjacent to the first photoelectric conversion element or the second photoelectric conversion element, and the third photoelectric conversion element collects all backscattering light intensities that do not pass any polarization light-converting structure.

In some embodiments, the first circularly polarized light is emitted into the biosample with an incident angle of −65 degrees to 65 degrees, and the detection device collects the backscattering light with a light collection angle of 20-65 degrees shifted from the incidence.

An embodiment of the present invention provides an optical system. The optical system includes a first light source and a circular polarizer for converting light emitted from the first light source to a first circularly polarized light. The optical system also includes a biosample illuminated by the first circularly polarized light to generate a backscattering light. The optical system further includes a detection device for detecting the backscattering light. The detection device includes a substrate having a first photoelectric conversion element and a second photoelectric conversion element. The detection device also includes a first filter disposed above the first photoelectric conversion element and a second filter disposed above the second photoelectric conversion element. The first photoelectric conversion element collects light with the same direction as the first circularly polarized light, and the second photoelectric conversion element collects light with opposite direction to the first circularly polarized light.

In some embodiments, the circular polarizer includes a linear polarizer and a quarter-wave plate disposed above the linear polarizer. The circular polarizer also includes a light-transmitting layer disposed between the linear polarizer and the quarter-wave plate.

In some embodiments, the circular polarizer includes metasurface.

In some embodiments, the first light source is disposed on the substrate, and the circular polarizer is disposed above the first light source and includes microlenses or metalens.

In some embodiments, the first light source emits light with wavelengths ranging from visible light to infrared light.

In some embodiments, the optical system further includes a second light source disposed on the substrate. The second light source emits light that has a different wavelength than light emitted from the first light source.

In some embodiments, the biosample is skin or organ tissue.

In some embodiments, the optical system further includes a collimator for collimating the first circularly polarized light.

An embodiment of the present invention provides a method for detecting the concentration of a specific substance in a biosample includes the following steps. The biosample with a circularly polarized light is illuminated with an incident angle of −65 degrees to +65 degrees. The detection device mentioned above is utilized to collect a backscattering light with a light collection angle of 20 degrees to 65 degrees shifted from the incidence. The first photoelectric conversion element is employed to collect a first signal intensity and the second photoelectric conversion element is employed to collect a second signal intensity. A degree of circular polarization is calculated using the formula of (the first signal intensity-the second signal intensity)/(the first signal intensity+the second signal intensity).

In some embodiments, the substrate further has a third photoelectric conversion element adjacent to the first photoelectric conversion element or the second photoelectric conversion element, and the method further includes the following steps. The third photoelectric conversion element is employed to collect a third signal intensity. A degree of circular polarization is calculated using the formula of (the first signal intensity-the second signal intensity)/(the third signal intensity).

### BRIEF DESCRIPTION OF THE DRAWINGS

The disclosure can be more fully understood from the following detailed description when read with the accompanying figures. It is worth noting that, in accordance with standard practice in the industry, various features are not drawn to scale. In fact, the dimensions of the various features may be arbitrarily increased or reduced for clarity of discussion.

FIG. 1 is a partial cross-sectional view illustrating the optical system 1 according to some embodiments of the present disclosure.

FIG. 2 is a three-dimensional schematic diagram illustrating the detection device according to some embodiments of the present disclosure.

FIG. 3 is a partial cross-sectional view illustrating the optical system according to some other embodiments of the present disclosure.

FIG. 4 is a three-dimensional schematic diagram illustrating the detection device according to some other embodiments of the present disclosure.

FIG. 5 is a partial cross-sectional view illustrating the detection device according to some other embodiments of the present disclosure.

FIG. 6 is a partial cross-sectional view illustrating the optical system according to some other embodiments of the present disclosure. FIG. 7 is a three-dimensional schematic diagram illustrating the detection device according to some other embodiments of the present disclosure.

FIG. 8 is a partial cross-sectional view illustrating the optical system according to some other embodiments of the present disclosure.

FIG. 9 is a three-dimensional schematic diagram illustrating the detection device according to some other embodiments of the present disclosure.

FIG. 10 is a partial cross-sectional view illustrating the optical system according to some other embodiments of the present disclosure.

FIG. 11 is a three-dimensional schematic diagram illustrating the detection device according to some other embodiments of the present disclosure.

### DETAILED DESCRIPTION OF THE INVENTION

The following disclosure provides many different embodiments, or examples, for implementing different features of the subject matter provided. Specific examples of components and arrangements are described below to simplify the present disclosure. These are, of course, merely examples and are not intended to be limiting. For example, a first feature is formed on a second feature in the description that follows may include embodiments in which the first feature and second feature are formed in direct contact, and may also include embodiments in which additional features may be formed between the first feature and second feature, so that the first feature and second feature may not be in direct contact.

It should be understood that additional steps may be implemented before, during, or after the illustrated methods, and some steps might be replaced or omitted in other embodiments of the illustrated methods.

Furthermore, spatially relative terms, such as “beneath,” “below,” “lower,” “on,” “above,” “upper” and the like, may be used herein for ease of description to describe one element or feature's relationship to other elements or features as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

In the present disclosure, the terms “about,” “approximately” and “substantially” typically mean +/−20% of the stated value, more typically +/−10% of the stated value, more typically +/−5% of the stated value, more typically +/−3% of the stated value, more typically +/−2% of the stated value, more typically +/−1% of the stated value and even more typically +/−0.5% of the stated value. The stated value of the present disclosure is an approximate value. That is, when there is no specific description of the terms “about,” “approximately” and “substantially”, the stated value includes the meaning of “about,” “approximately” or “substantially”.

Unless otherwise defined, all terms (including technical and scientific terms) used herein have the same meaning as commonly understood by one of ordinary skill in the art to which this disclosure belongs. It should be understood that terms such as those defined in commonly used dictionaries should be interpreted as having a meaning that is consistent with their meaning in the context of the relevant art and will not be interpreted in an idealized or overly formal sense unless expressly so defined in the embodiments of the present disclosure.

The present disclosure may repeat reference numerals and/or letters in following embodiments. This repetition is for the purpose of simplicity and clarity and does not in itself dictate a relationship between the various embodiments and/or configurations discussed.

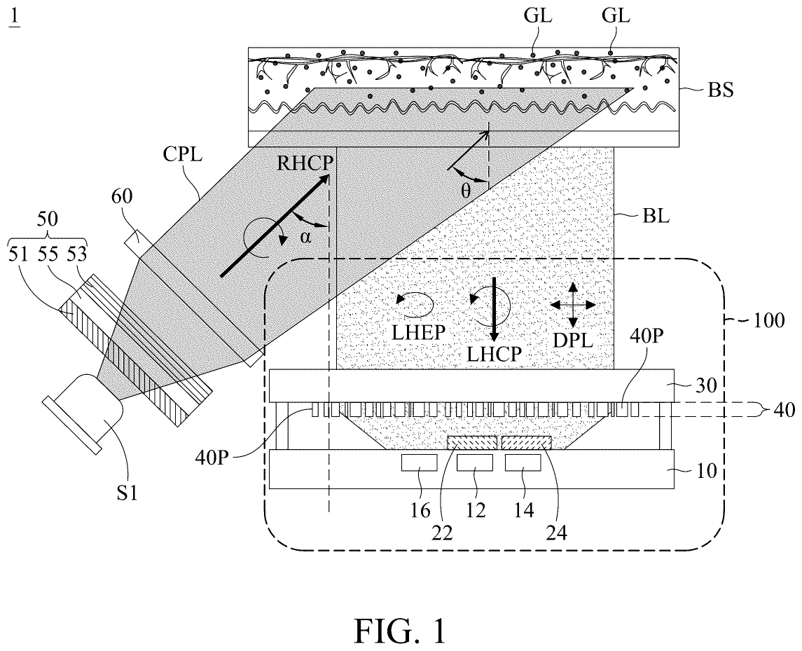

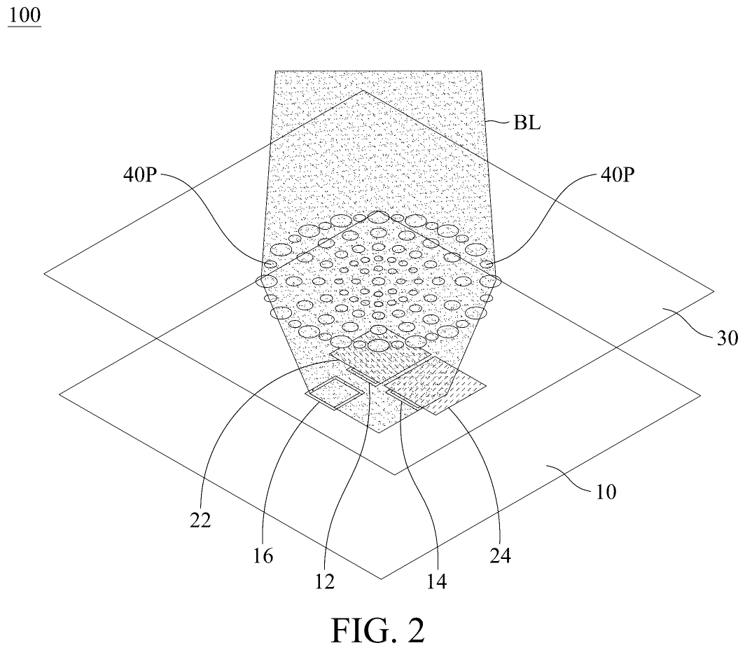

FIG. 1 is a partial cross-sectional view illustrating the optical system 1 according to some embodiments of the present disclosure. FIG. 2 is a three-dimensional schematic diagram illustrating the detection device 100 according to some embodiments of the present disclosure. It should be noted that some components have been omitted in FIG. 1 and FIG. 2 for the sake of brevity.

Referring to FIG. 1, in some embodiments, the optical system 1 includes a light source S1 and a circular polarizer 50 for converting light emitted from the light source S1 to a circularly polarized light CPL. In some embodiments, the light source S1 emits light with wavelengths ranging from visible light to infrared light. For example, light emitted from the light source S1 may have a wavelength of about 460 nm, about 532 nm, about 660 nm, about 785 nm, or about 940 nm, but the present disclosure is not limited thereto.

As shown in FIG. 1, in some embodiments, the circular polarizer 50 includes a linear polarizer 51 and a quarter-wave plate (λ/4 plate) 53 disposed above the linear polarizer 51. The combination of the linear polarizer 51 and the quarter-wave plate 53 may be used as a circular polarizer. For example, the circular polarizer 50 may convert light emitted from the light source S1 to right-handed circular polarization light RHCP, but the present disclosure is not limited thereto. Moreover, the circular polarizer 50 further includes a light-transmitting layer 55 disposed between the linear polarizer 51 and the quarter-wave plate 53, but the present disclosure is not limited thereto. In some other embodiments, the circular polarizer 50 includes a (single) metasurface.

Referring to FIG. 1, in some embodiments, the optical system 1 includes a biosample BS illuminated by the circularly polarized light CPL to generate a backscattering light BL. In some embodiments, the biosample BS is skin or organ tissue. For example, the biosample BS may be a palm, a wrist, or a finger of hand of a human body, but the present disclosure is not limited thereto. As shown in FIG. 1, in some embodiments, the circularly polarized light CPL is emitted into the biosample BS with an incident angle θ of about −65 degrees to about +65 degrees. Here, the incident angle θ is defined between the normal line of biosample BS and the incident light (i.e., the circularly polarized light CPL).

Brewster's angle, also called the polarization angle, is the incident angle where an unpolarized electromagnetic wave separates into a vertically polarized wave that transmits through the surface, leaving only the horizontal component in the reflected wave. Brewster's angle is directly related to the refractive indices of the two media involved. It is the angle of incidence at which light with a particular polarization is perfectly transmitted through a transparent dielectric surface, with no reflection.

When the incident light is linearly polarized and the incident angle is greater than Brewster's angle, several things will occur. The horizontally polarized light (parallel to the plane of incidence) will be partially reflected and partially refracted. The vertically polarized light (perpendicular to the plane of incidence) will also be partially reflected and partially refracted, but its reflectance will increase with the incident angle.

When the incidence light is polarized by a linear polarizer in an angle not parallel to the plane of the light pathway and the incident angle is larger than Brewster's angle, a few portion of polarized light is more reflected and not transmitted into the biosamples. It cause the ratio of the polarized light different in the biosample when the incident angle is larger than Brewster's angle. In order to avoid this phenomenon and easy to arrange the incident light, the incident angle θ should be smaller than Brewster's angle, usually smaller than about 60-65 degree.

Brewster's angle can be calculated using arctan (n2/n1), where n1 is the refractive index of the initial medium (e.g., air) and n2 is the refractive index of the medium that the light is entering (e.g., skin or other tissue with refractive index of about 1.4-1.55). Due to Brewster's angle varies depending on the refractive indexes of the two interfaces, difference incident light wavelength may have different largest incident angle limitation.

As shown in FIG. 1, in some embodiments, the optical system 1 further includes a collimator 60 for collimating the circularly polarized light CPL. In some other embodiments, the collimator 60 may be omitted. For example, the collimator 60 may be placed between the circular polarizer 50 and the biosample BS, so as to ensure the circularly polarized light CPL is collimated into the biosample BS.

Referring to FIG. 1, in some embodiments, the optical system 1 includes a detection device 100 for detecting the backscattering light BL. In other words, the detection device 100 may be used for detecting the backscattering light BL that is generated after the circularly polarized light CPL is emitted into the biosample BS. After entering the biosample BS, the circularly polarized light CPL may be mostly absorbed and partially reflected/scattered in an inversed chirality and unpolarized light due to the collision of the interface and the components within the biosample BS. That is, the backscattering light BL may include circularly polarized light (e.g., left-handed circular polarization light LHCP) and depolarized light DPL.

Referring to FIG. 1 and FIG. 2, in some embodiments, the detection device 100 includes a substrate 10. For example, the substrate 10 may include a semiconductor substrate. The substrate 10 may be made of an elementary semiconductor (e.g., silicon or germanium), a compound semiconductor (e.g., silicon carbide (SiC), gallium arsenic (GaAs), indium arsenide (InAs), or indium phosphide (InP)), an alloy semiconductor (e.g., silicon germanium (SiGe), silicon germanium carbide (SiGeC), gallium arsenic phosphide (GaAsP), or gallium indium phosphide (GaInP)), any other applicable semiconductor, or a combination thereof. Moreover, the substrate 10 may be a semiconductor-on-insulator (SOI) substrate. The semiconductor-on-insulator substrate may include a bottom substrate, a buried oxide layer disposed on the bottom substrate, and a semiconductor layer disposed on the buried oxide layer. Furthermore, the substrate 10 may be a semiconductor wafer (e.g., a silicon wafer, or any other applicable semiconductor wafer).

The substrate 10 may include various p-type doped regions and/or n-type doped regions formed by a process such as an ion implantation process and/or a diffusion process. For example, the doped regions may be configured to form a transistor, a photodiode, and/or a light-emitting diode, but the present disclosure is not limited thereto.

The substrate 10 may also include various isolation features to separate various device regions in the substrate 10. For example, the isolation features may include a shallow trench isolation (STI) feature, but the present disclosure is not limited thereto. The formation of a shallow trench isolation (STI) feature may include etching a trench in the substrate 10 and filling in the trench with insulating materials (e.g., silicon oxide, silicon nitride, or silicon oxynitride). The filled trench may have a multi-layer structure, such as a thermal oxide liner layer with silicon nitride filling the trench. A chemical mechanical polishing (CMP) process may be performed to polish back excessive insulating materials and planarize the top surface of the isolation features.

Moreover, the substrate 10 may include various conductive features (e.g., conductive lines or vias). For example, the conductive features may be made of aluminum (Al), copper (Cu), tungsten (W), an alloy thereof, any other applicable conductive material, or a combination thereof. As shown in FIG. 1 and FIG. 2, in some embodiments, the detection device 100 includes a first photoelectric conversion element 12 and a second photoelectric conversion element 14. In some embodiments, the first photoelectric conversion element 12 collects light with the same polarization direction as the circularly polarized light CPL, and the second photoelectric conversion element 14 collects light with opposite polarization direction to the circularly polarized light CPL.

When unpolarized light passes through a linear polarizer (e.g., linear polarizer 51), it becomes linearly polarized. If this linearly polarized light then passes through a quarter-wave plate (λ/4 plate, e.g., quarter-wave plate 53) with its fast axis at a 45-degree angle to the polarization direction, the light is converted into circularly polarized light (e.g., circularly polarized light CPL). For example, let's assume the linear polarizer produces light polarized at an angle of 0 degrees (horizontal polarization). When this horizontally polarized light passes through a quarter-wave plate whose fast axis is oriented at 45 degrees clockwise from the polarization direction (0 degrees), the resulting light will be right-handed circularly polarized (e.g., right-handed circular polarization light RHCP). Conversely, if the quarter-wave plate's fast axis is oriented at 45 degrees counterclockwise from the polarization direction (0 degrees), the resulting light will be left-handed circularly polarized. Therefore, if the linear polarizer sets the polarization at 0 degrees (horizontal), and the quarter-wave plate's fast axis is rotated 45 degrees clockwise, it will produce right-handed circularly polarized light. If the linear polarizer sets the polarization at 0 degrees (horizontal), and the quarter-wave plate's fast axis is rotated 45 degrees counterclockwise, it will produce left-handed circularly polarized light.

As shown in FIG. 1 and FIG. 2, in some embodiments, the detection device 100 includes a first filter 22 and a second filter 24, the first filter 22 is disposed above the first photoelectric conversion element 12, and the second filter 24 is disposed above the second photoelectric conversion element 14. For example, the first filter 22 may let only right-handed circular polarization light RHCP, so that the first photoelectric conversion element 12 collects light with the same polarization direction as the right-handed circularly polarized light RHCP. The second filter 24 may let only left-handed circular polarization light LHCP pass, so that the second photoelectric conversion element 14 collects light with opposite polarization direction to the right-handed circularly polarized light RHCP (i.e., the second photoelectric conversion element 14 collects light with the same polarization direction as the left-handed circularly polarized light LHCP).

As shown in FIG. 1 and FIG. 2, in some embodiments, the substrate 10 further has a third photoelectric conversion element 16 adjacent to the first photoelectric conversion element 12 or the second photoelectric conversion element 14. Moreover, the third photoelectric conversion element 16 collects all backscattering light intensities that do not pass any polarization light-converting structure.

As shown in FIG. 1 and FIG. 2, in some embodiments, the detection device 100 further includes a cover plate 30 disposed over the first filter 22 and the second filter 24. For example, the cover plate 30 may include glass, and have a thickness of about 50 μm to about 1000 μm, but the present disclosure is not limited thereto.

Moreover, in some embodiments, the detection device 100 further includes a light-condensing structure 40 disposed on the cover plate 30. In this embodiment, the light-condensing structure 40 includes microlenses or metalens 40P. For example, the light-condensing structure 40 includes tantalum pentoxide (Ta₂O₅), titanium dioxide (TiO₂), niobium oxide (Nb₂O₅), silicon nitride (Si₃N₄), silver (Ag), gallium nitride (GaN), and silicon (Si), which may have a function of focusing the backscattering light (BL) to the first photoelectric conversion element 12, the second photoelectric conversion element 14, and the third photoelectric conversion element 16.

Taking the biosample BS as human skin and the circularly polarized light CPL is right-handed circular polarization light RHCP as an example, if there is no glucose (or any other chiral material) in the biosample BS, the major signals in the backscattering light BL are from left-handed circular polarization light LHCP and depolarized light DPL. In this example, the first photoelectric conversion element 12 collects signal intensities of vertical weighting of depolarized light DPL (which will be referred to as PD12), the second photoelectric conversion element 14 collects signal intensities of horizontal weighting of depolarized light DPL and left-handed circular polarization light LHCP (which will be referred to as PD14), and the third photoelectric conversion element 16 may collect signal intensities of all backscattering light BL that do not pass any polarization light-converting structure (which will be referred to as PD16). Moreover, the degree of circular polarization (DOP) may follow the formula: DOP=(PD12−PD14)/(PD12+PD14) or (PD12−PD14)/PD16. Here, if DOP is a negative number, it indicates a rotational inversion which means a reflection light of a right-handed circular polarization light RHCP changes to a left-handed circular polarization light LHCP. It should be noted that depolarized light DPL has equal vertical and horizontal weighting.

As shown in FIG. 1, if there is more glucose GL (e.g., d-glucose) (or any other chiral material) in the biosample BS, the more glucose, the more left-handed circular polarization light LHCP and the less depolarized light DPL in the backscattering light (BL), and the increased portion of the more left-handed circular polarization light LHCP trends to be left-handed elliptic polarization light LHEP (due to d-glucose has different absorption capability to different polarized lights). In this example, the first photoelectric conversion element 12 collects signal intensities of vertical weighting of depolarized light DPL and left-handed elliptic polarization light LHEP (which will be referred to as PD12), the second photoelectric conversion element 14 collects signal intensities of horizontal weighting of depolarized light DPL and left-handed elliptic polarization light LHEP (which will be referred to as PD14), and the third photoelectric conversion element 16 may collect signal intensities of all backscattering light BL (which will be referred to as PD16). Moreover, the degree of circular polarization (DOP) may follow the formula: DOP=(PD12−PD14)/(PD12+PD14) or (PD12−PD14)/PD16. Here, if DOP is a negative number, it indicates a rotational inversion which means a reflection light of a right-handed circular polarization light RHCP changes to a left-handed circular polarization light LHCP. It should be noted that horizontal weighting of left-handed elliptic polarization light LHEP is higher than vertical weighting of left-handed elliptic polarization light LHEP. Besides, the difference of horizontal weighting and vertical weighting of left-handed elliptic polarization light LHEP is proportional to the glucose concentration. Therefore, the glucose concentration may be measured by the first photoelectric conversion element 12 and the second photoelectric conversion element 14 (and in some embodiments with the third photoelectric conversion element 16).

In some embodiments, a method for detecting the concentration of a specific substance (e.g., d-glucose) in a biosample BS (e.g., human skin) includes the following steps. The biosample BS with a circularly polarized light CPL is illuminated with an incident angle θ of about −65 degrees to about +65 degrees. Then, a detection device (e.g., detection device 100) is utilized to collect a backscattering light BL with a light collection angle a of about 20 degrees to about 65 degrees shifted from the incidence. Here, the light collection angle a is defined as the angle between the forward direction of the circularly polarized light CPL and the normal line of the detection device 100 as shown in FIG. 1. Then, the first photoelectric conversion element 12 is employed to collect a first signal intensity PD12 and the second photoelectric conversion element 14 is employed to collect a second signal intensity PD14. Then, a degree of circular polarization DOP is calculated using the formula of (first signal intensity-second signal intensity)/(first signal intensity+second signal intensity) (i.e., (PD12−PD14)/(PD12+PD14)).

Alternately, the third photoelectric conversion element 16 is employed to collect a third signal intensity PD16, and the degree of circular polarization DOP is calculated using the formula of (first signal intensity-second signal intensity)/(third signal intensity) (i.e., (PD12−PD14)/PD16).

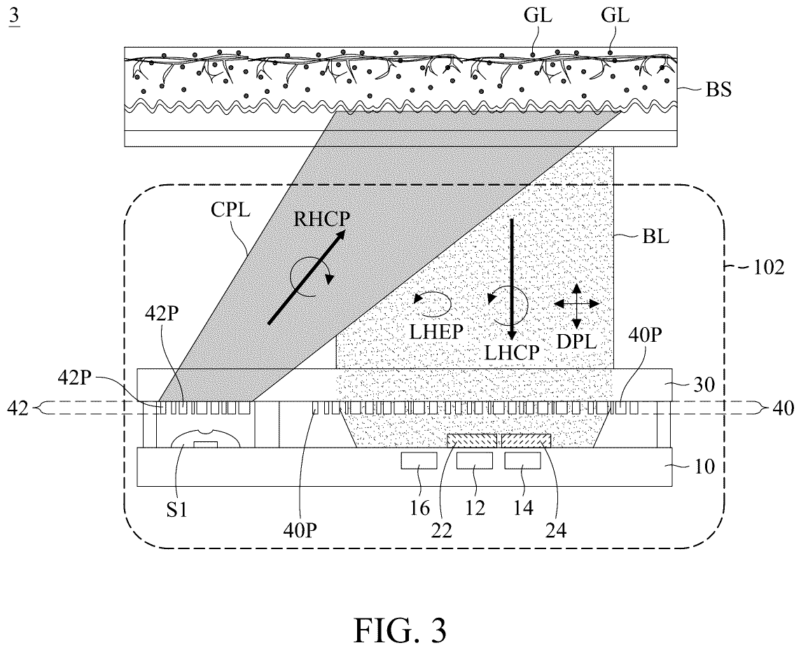

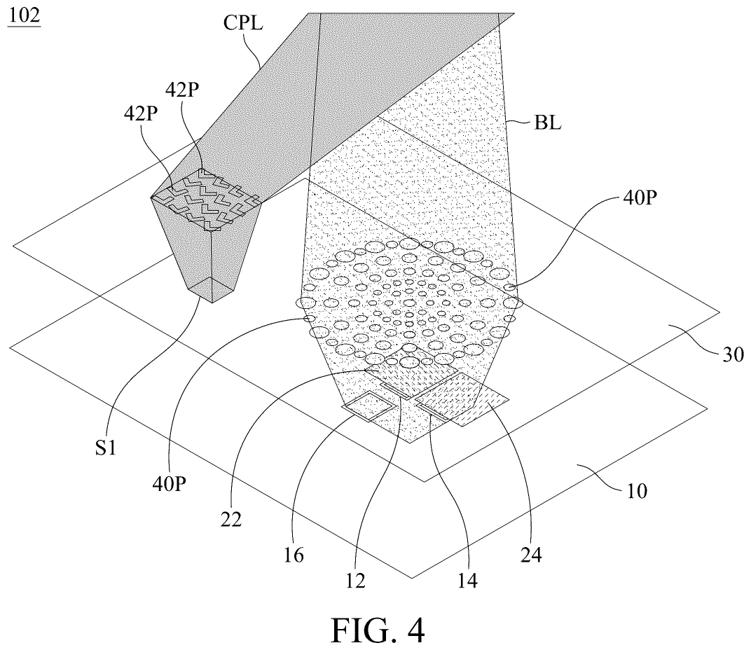

FIG. 3 is a partial cross-sectional view illustrating the optical system 3 according to some other embodiments of the present disclosure. FIG. 4 is a three-dimensional schematic diagram illustrating the detection device 102 according to some other embodiments of the present disclosure. It should be noted that some components have been omitted in FIG. 3 and FIG. 4 for the sake of brevity.

As shown in FIG. 3 and FIG. 4, in some embodiments, the optical system 3 includes a light source S1 and a circular polarizer (e.g., polarization light-converting structure 42) that is made by metasurface structure (including a plurality of nanoelements in various shape and orientation, the nanoelement may include tantalum pentoxide (Ta₂O₅), titanium dioxide (TiO₂), niobium oxide (Nb₂O₅), silicon nitride (Si₃N₄), silver (Ag), gallium nitride (GaN), and silicon (Si)) for converting light emitted from the light source S1 to a circularly polarized light CPL (that could be right-handed circular polarization light RHCP or left-handed circular polarization light LHCP, here we use right-handed circular polarization light RHCP as an example). The optical system 3 also includes a biosample BS illuminated by the circularly polarized light CPL to generate a backscattering light BL. The optical system 3 further includes a detection device 102 for detecting the backscattering light BL. The detection device 102 includes a substrate 10 having a first photoelectric conversion element 12 and a second photoelectric conversion element 14. The detection device 102 also includes a first filter 22 disposed above the first photoelectric conversion element 12 and a second filter disposed 24 above the second photoelectric conversion element 14. The first photoelectric conversion element 12 collects light with the same polarization direction as the circularly polarized light CPL, and the second photoelectric conversion element 14 collects light with opposite polarization direction to the circularly polarized light CPL.

As show in FIG. 3 and FIG. 4, similarly, the detection device 102 includes a cover plate 30 disposed over the first filter 22 and the second filter 24, and the detection device 102 also includes a light-condensing structure 40 disposed on the cover plate 30. Moreover, the substrate 10 further has a third photoelectric conversion element 16 adjacent to the first photoelectric conversion element 12 or the second photoelectric conversion element 14 to collect all backscattering light intensities that do not pass any polarization light-converting structure.

As show in FIG. 3 and FIG. 4, in this embodiment, the light source S1 is disposed on the substrate 10, and the detection device 102 further includes a polarization light-converting structure 42 disposed above the light source S1. The circularly polarized light CPL is generated from the light source S1 through the polarization light-converting structure 42. In other words, the polarization light-converting structure 42 may be used as a circular polarizer for converting light emitted from the light source S1 to the circularly polarized light CPL, but the present disclosure is not limited thereto. As show in FIG. 3 and FIG. 4, in some embodiments, the polarization light-converting structure 42 includes metasurface 42P. The size and/or the arrangement of metasurface 42P may be different from the size and/or the arrangement of microlenses or metalens 40P. In some other embodiments, the polarization light-converting structure 42 may have a structure similar to the circular polarizer 50 shown in FIG. 1. That is, the polarization light-converting structure 42 includes a linear polarizer and a quarter-wave plate (λ/4 plate) disposed above the linear polarizer. Moreover, the polarization light-converting structure 42 further includes a light-transmitting layer disposed between the linear polarizer and the quarter-wave plate.

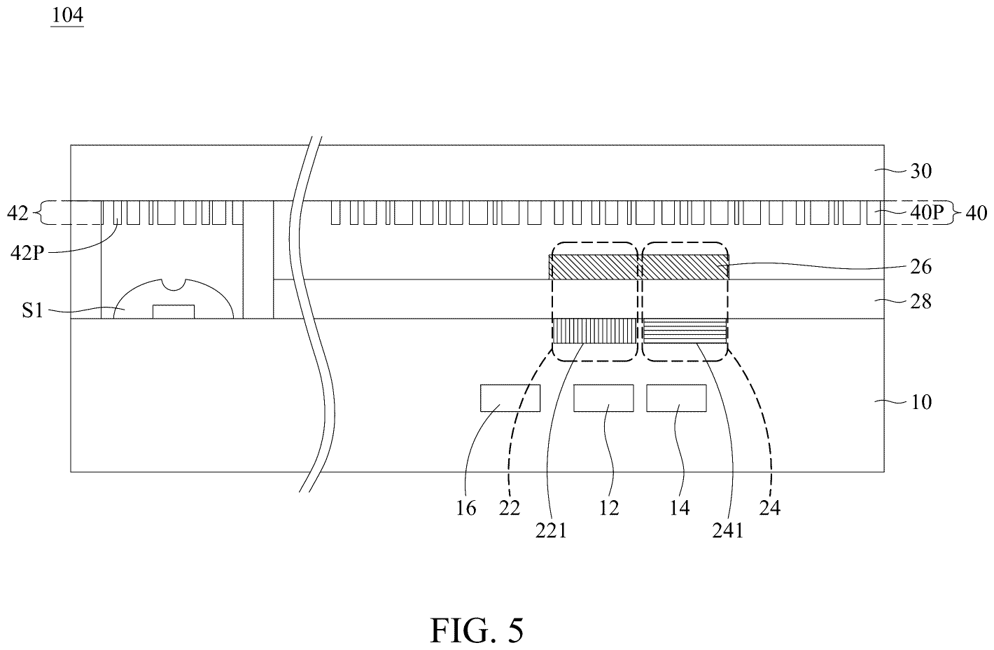

FIG. 5 is a partial cross-sectional view illustrating the detection device 104 according to some other embodiments of the present disclosure. It should be noted that some components of the detection device 104 have been omitted in FIG. 5 for the sake of brevity.

As shown in FIG. 5, in some embodiments, the detection device 104 further includes a first linear polarizer 221, a second linear polarizer 241, a quarter-wave plate 26, and a light-transmitting layer 28. The first linear polarizer 221 is disposed above the first photoelectric conversion element 12. The second linear polarizer 241 is disposed above the second photoelectric conversion element 14, and the second linear polarizer is shifted 90 degrees from the first linear polarizer 221. The quarter-wave plate 26 is disposed above the first linear polarizer 221 and the second linear polarizer 241. The light-transmitting layer 28 disposed between the first linear polarizer 221 (or the second linear polarizer 241) and the quarter-wave plate 26.

In more detail, the first linear polarizer 221 (or the second linear polarizer 241) and the quarter-wave plate 26 are disposed on two opposite sides of the light-transmitting layer 28. In this embodiment, the first linear polarizer 221, the light-transmitting layer 28, and the quarter-wave plate 26 form the first filter 22, and the second linear polarizer 241, the light-transmitting layer 28, and the quarter-wave plate 26 form the second filter 24.

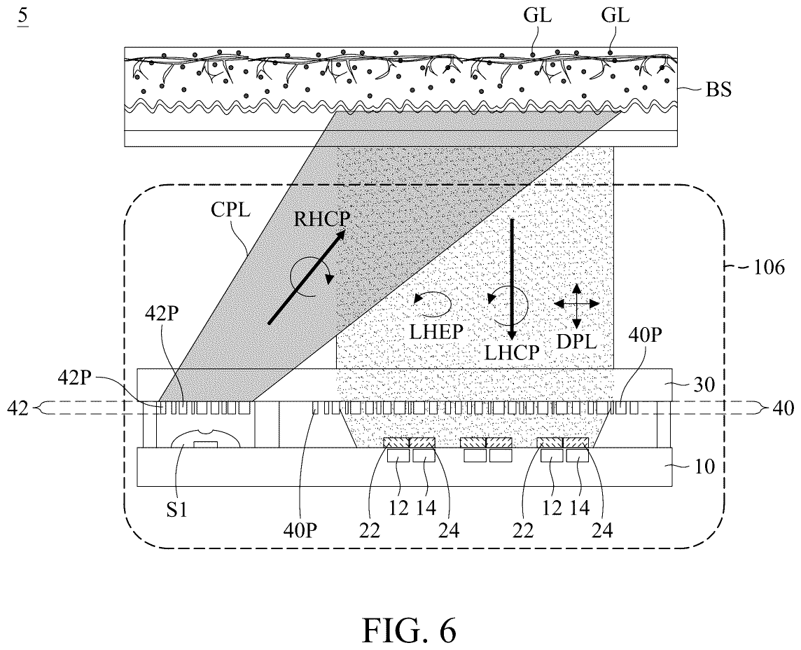

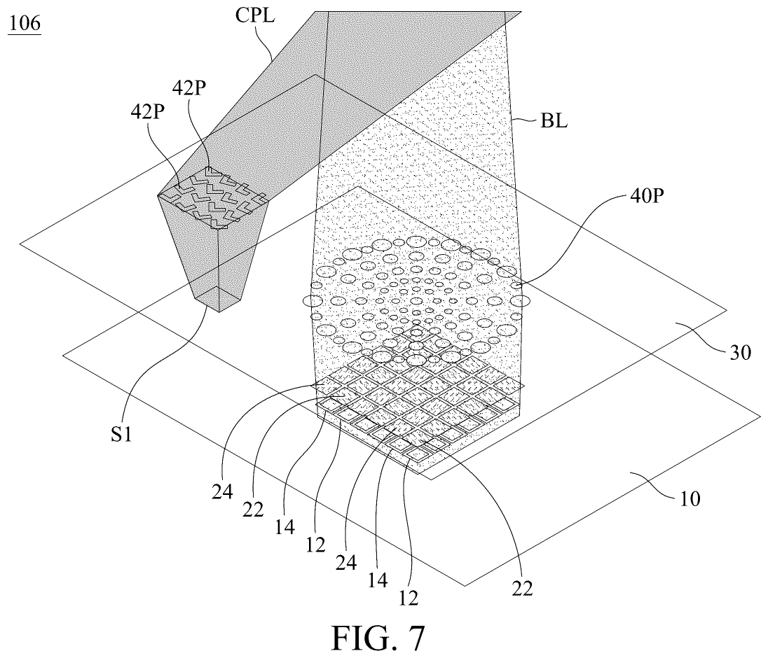

FIG. 6 is a partial cross-sectional view illustrating the optical system 5 according to some other embodiments of the present disclosure. FIG. 7 is a three-dimensional schematic diagram illustrating the detection device 106 according to some other embodiments of the present disclosure. It should be noted that some components have been omitted in FIG. 6 and FIG. 7 for the sake of brevity.

As shown in FIG. 6 and FIG. 7, there is no third photoelectric conversion element 16 in the detection device 106. In this embodiment, the detection device 106 includes a plurality of first photoelectric conversion elements 12 and a plurality of second photoelectric conversion elements 14. As shown in FIG. 7, the first photoelectric conversion elements 12 and the second photoelectric conversion elements 14 are arranged in an array. Moreover, the detection device 106 also includes a plurality of first filters 22 disposed above the first photoelectric conversion elements 12 and a plurality of second filters 24 disposed above the second photoelectric conversion elements 14. That is, the first filters 22 and the second filters 24 are also arranged in an array as show in FIG. 7, but the present disclosure is not limited thereto.

Moreover, the value of degree of circular polarization DOP measured from the photoelectric conversion elements (e.g., first photoelectric conversion elements 12 and second photoelectric conversion elements 14) are mapped to reconstruct a DOP image of the biosample BS. For example, if the biosample BS is a tissue section to analyze pathological images, the biosample BS may be placed above the detection device 106 in FIG. 7. The signal within the field of view can be focused through the microlenses or metalens 40P onto the corresponding array below. Every two pixels (i.e., one first photoelectric conversion element 12 and one second photoelectric conversion element 14) can be used to calculate the degree of circular polarization (DOP), and the entire array can be used to compute the DOP values for adjacent regions, which can then be mapped into a DOP numerical image.

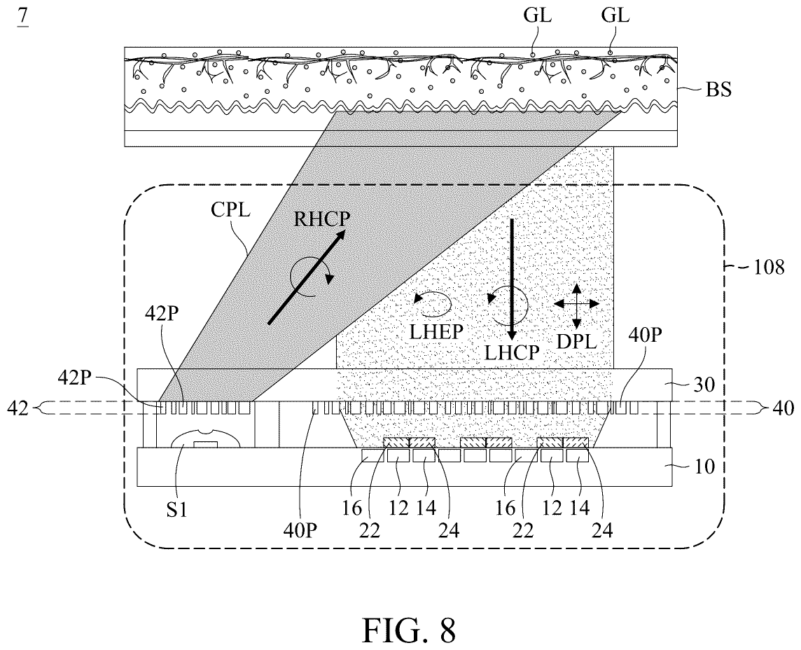

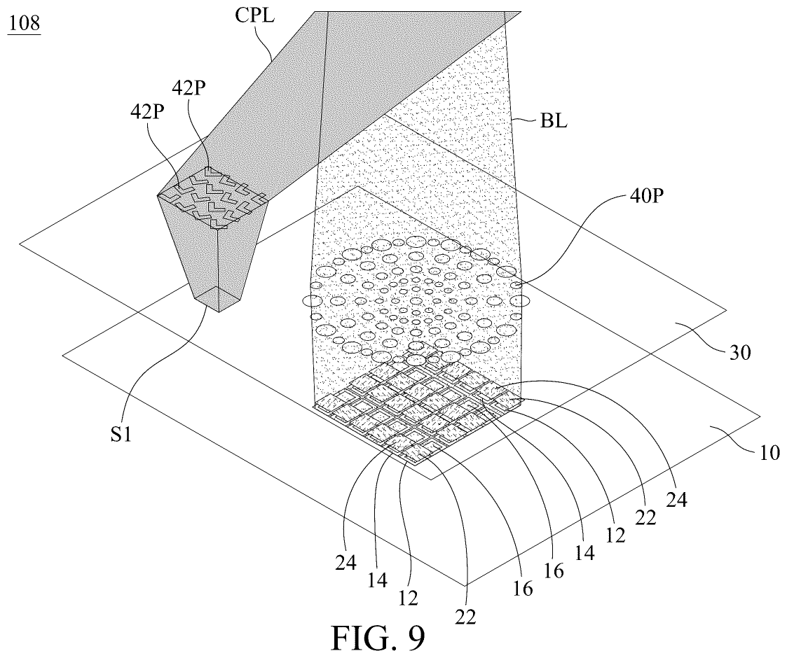

FIG. 8 is a partial cross-sectional view illustrating the optical system 7 according to some other embodiments of the present disclosure. FIG. 9 is a three-dimensional schematic diagram illustrating the detection device 108 according to some other embodiments of the present disclosure. It should be noted that some components have been omitted in FIG. 8 and FIG. 9 for the sake of brevity.

As shown in FIG. 8 and FIG. 9, similarly, the detection device 108 includes a plurality of first photoelectric conversion elements 12 and a plurality of second photoelectric conversion elements 14. Moreover, the detection device 108 also includes a plurality of first filters 22 disposed above the first photoelectric conversion elements 12 and a plurality of second filters 24 disposed above the second photoelectric conversion elements 14. In this embodiments, the detection device 108 further includes a plurality of third photoelectric conversion elements 16 disposed adjacent to the first photoelectric conversion elements 12 or the second photoelectric conversion elements 14. As shown in FIG. 9, in this embodiment, the first photoelectric conversion elements 12, the second photoelectric conversion elements 14, and the third photoelectric conversion elements 16 are arranged in an array. Besides, there is no filters disposed above the third photoelectric conversion elements 16.

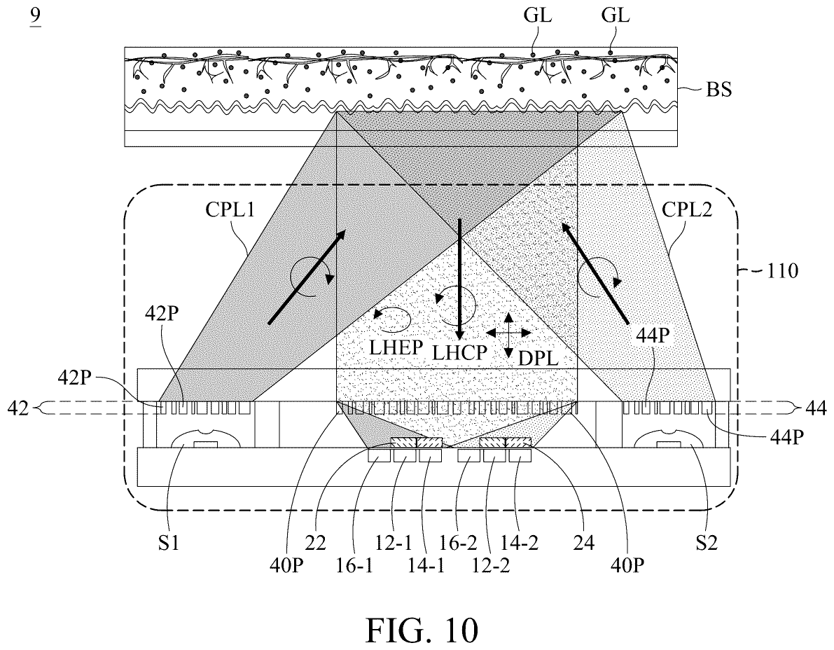

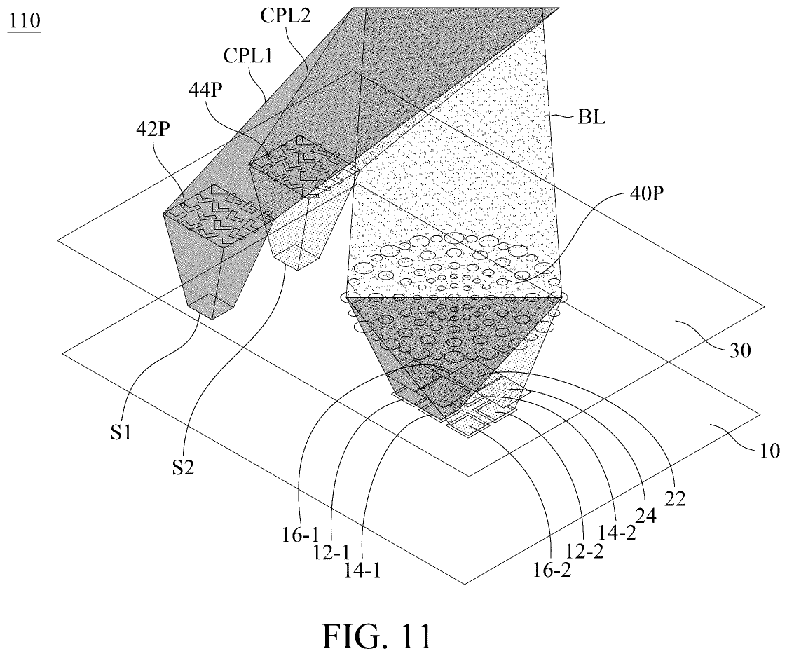

FIG. 10 is a partial cross-sectional view illustrating the optical system 9 according to some other embodiments of the present disclosure. FIG. 11 is a three-dimensional schematic diagram illustrating the detection device 110 according to some other embodiments of the present disclosure. It should be noted that some components have been omitted in FIG. 10 and FIG. 11 for the sake of brevity.

As shown in FIG. 10 and FIG. 11, in some embodiments, the detection device 110 includes another light source S2 disposed on the substrate 10. As shown in FIG. 11, the light source S1 and the light source S2 may be, for example, arranged on the same side of the substrate 10, but the present disclosure is not limited thereto. In other examples, the light source S1 and the light source S2 may be arranged on opposite sides of the substrate 10. In some embodiments, the detection device 110 also includes a polarization light-converting structure 44 disposed above the light source S2. Similarly, in some embodiments, the polarization light-converting structure 44 includes metasurface 44P. Moreover, in some other embodiments, the polarization light-converting structure 44 may have a structure similar to the circular polarizer 50 shown in FIG. 1. That is, the polarization light-converting structure 4 includes a linear polarizer and a quarter-wave plate (λ/4 plate) disposed above the linear polarizer. Moreover, the polarization light-converting structure 44 further includes a light-transmitting layer disposed between the linear polarizer and the quarter-wave plate.

As show in FIG. 10 and FIG. 11, in this embodiment, a circularly polarized light CPL2 having a different wavelength than the circularly polarized light CPL1 is generated from the light source S2 through the polarization light-converting structure 44. In other words, the polarization light-converting structure 42 may be used as a circular polarizer for converting light emitted from the light source S1 to the circularly polarized light CPL1, while the polarization light-converting structure 44 may be used as a circular polarizer for converting light emitted from the light source S2 to the circularly polarized light CPL2, but the present disclosure is not limited thereto.

For example, the light source S1 may emits green light, and the light source S2 may emits red light. Since different lights have different penetration depths, lights from light sources S1 and S2 can be applied to many different human races. It can also be equipped with reflection photoplethysmography (PPG) (e.g., green light from the light source S1) and blood oxygen detection (e.g., green light from the light source S1 and red light from the light source S2).

As show in FIG. 10 and FIG. 11, in this embodiment, there is at least one set of first photoelectric conversion element 12-1 that collects light with the same polarization direction as the circularly polarized light CPL1, second photoelectric conversion element 14-1 that collects light with opposite polarization direction to the circularly polarized light CPL1, and third photoelectric conversion element 16-1 that collects all backscattering light that do not pass any polarization light-converting structure from the circularly polarized light CPL1. Moreover, there is at least one set of first photoelectric conversion element 12-2 that collects light with the same polarization direction as the circularly polarized light CPL2, second photoelectric conversion element 14-2 that collects light with opposite polarization direction to the circularly polarized light CPL2, and third photoelectric conversion element 16-2 that collects all backscattering light that do not pass any polarization light-converting structure from the circularly polarized light CPL2. However, the present disclosure is not limited thereto. In some other embodiments, there is no third photoelectric conversion element 16-1 or 16-2.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure. Therefore, the scope of protection should be determined through the claims. In addition, although some embodiments of the present disclosure are disclosed above, they are not intended to limit the scope of the present disclosure.

Reference throughout this specification to features, advantages, or similar language does not imply that all of the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the disclosure.

What is claimed is:

1. A detection device for detecting a backscattering light that is generated after a first circularly polarized light is emitted into a biosample, comprising:
a substrate having a first photoelectric conversion element and a second photoelectric conversion element;
a first filter disposed above the first photoelectric conversion element; and
a second filter disposed above the second photoelectric conversion element;
wherein the first photoelectric conversion element collects light with the same polarization direction as the first circularly polarized light, and the second photoelectric conversion element collects light with opposite polarization direction to the first circularly polarized light.

2. The detection device as claimed in claim 1, further comprising:
a cover plate disposed over the first filter and the second filter; and
a light-condensing structure disposed on the cover plate.

3. The detection device as claimed in claim 2, wherein the light-condensing structure comprises microlenses or metalens.

4. The detection device as claimed in claim 1, further comprising:
a first linear polarizer disposed above the first photoelectric conversion element;
a second linear polarizer disposed above the second photoelectric conversion element, wherein the second linear polarizer is shifted 90 degrees from the first linear polarizer;
a quarter-wave plate disposed above the first linear polarizer and the second linear polarizer; and
a light-transmitting layer disposed between the first linear polarizer or the second linear polarizer and the quarter-wave plate,
wherein the first linear polarizer, the light-transmitting layer, and the quarter-wave plate form the first filter, and the second linear polarizer, the light-transmitting layer, and the quarter-wave plate form the second filter.

5. The detection device as claimed in claim 1, further comprising:
a first light source disposed on the substrate; and
a first polarization light-converting structure disposed above the first light source,
wherein the first circularly polarized light is generated from the first light source through the first polarization light-converting structure.

6. The detection device as claimed in claim 5, wherein the first polarization light-converting structure comprises metasurface.

7. The detection device as claimed in claim 5, wherein the first polarization light-converting structure comprises:
a linear polarizer;
a quarter-wave plate disposed above the linear polarizer; and
a light-transmitting layer disposed between the linear polarizer and the quarter-wave plate.

8. The detection device as claimed in claim 5, further comprising:
a second light source disposed on the substrate; and
a second polarization light-converting structure disposed above the second light source,
wherein a second circularly polarized light having a different wavelength than the first circularly polarized light is generated from the second light source through the second polarization light-converting structure.

9. The detection device as claimed in claim 8, wherein the second polarization light-converting structure comprises metasurface.

10. The detection device as claimed in claim 8, wherein the second polarization light-converting structure comprises:
a linear polarizer;
a quarter-wave plate disposed above the linear polarizer; and
a light-transmitting layer disposed between the linear polarizer and the quarter-wave plate.

11. The detection device as claimed in claim 1, wherein there are a plurality of first photoelectric conversion elements and a plurality of second photoelectric conversion elements that are arranged in an array, and there are a plurality of first filters disposed above the plurality of first photoelectric conversion elements and a plurality of second filters disposed above the plurality of second photoelectric conversion elements.

12. The detection device as claimed in claim 1, wherein the substrate further has a third photoelectric conversion element adjacent to the first photoelectric conversion element or the second photoelectric conversion element, and the third photoelectric conversion element collects all backscattering light intensities that do not pass any polarization light-converting structure.

13. The detection device as claimed in claim 1, wherein the first circularly polarized light is emitted into the biosample with an incident angle of −65 degrees to +65 degrees, and the detection device collects the backscattering light with a light collection angle of 20-65 degrees shifted from an incidence.

14. An optical system, comprising:
a first light source;
a circular polarizer for converting light emitted from the first light source to a first circularly polarized light;
a biosample illuminated by the first circularly polarized light to generate a backscattering light; and
a detection device for detecting the backscattering light, wherein the detection device comprises:
a substrate having a first photoelectric conversion element and a second photoelectric conversion element;
a first filter disposed above the first photoelectric conversion element; and
a second filter disposed above the second photoelectric conversion element;
wherein the first photoelectric conversion element collects light with the same direction as the first circularly polarized light, and the second photoelectric conversion element collects light with opposite direction to the first circularly polarized light.

15. The optical system as claimed in claim 14, wherein the circular polarizer comprises:
a linear polarizer;
a quarter-wave plate disposed above the linear polarizer;
a light-transmitting layer disposed between the linear polarizer and the quarter-wave plate; and
a collimator for collimating the first circularly polarized light,
wherein the circular polarizer comprises metasurface.

16. The optical system as claimed in claim 14, wherein the first light source is disposed on the substrate, and the circular polarizer is disposed above the first light source and comprises metasurface.

17. The optical system as claimed in claim 14, wherein the first light source emits light with wavelengths ranging from visible light to infrared light, and the biosample is skin or organ tissue.

18. The optical system as claimed in claim 14, further comprising:
a second light source disposed on the substrate, wherein the second light source emits light that has a different wavelength than light emitted from the first light source.

19. A method for detecting the concentration of a specific substance in a biosample, comprising:
illuminating the biosample with a circularly polarized light with an incident angle of −65 degrees to 65 degrees;
utilizing a detection device as claimed in claim 1 to collect a backscattering light with a light collection angle of 20 degrees to 65 degrees shift from an incidence;
employing the first photoelectric conversion element to collect a first signal intensity and the second photoelectric conversion element to collect a second signal intensity; and
calculating a degree of circular polarization using the formula of (first signal intensity−second signal intensity)/(first signal intensity+second signal intensity).

20. The method for detecting the concentration of a specific substance in a biosample as claimed in claim 19, wherein the substrate further has a third photoelectric conversion element adjacent to the first photoelectric conversion element or the second photoelectric conversion element, and the method further comprises:
employing the third photoelectric conversion element to collect a third signal intensity; and
calculating a degree of circular polarization using the formula of (the first signal intensity−the second signal intensity)/(the third signal intensity).
